(19) United States
(12) Patent Application Publication
(10) Pub. No.: US 2016/0166702 A1
(43) Pub. Date: Jun. 16, 2016

(54) EXTENDED RELEASE AQUEOUS SUSPENSION OF METHYLPHENIDATE OR SALTS THEREOF

(71) Applicant: Gavis Pharmaceuticals, Somerset, NJ (US)

(72) Inventors: Prabagaran Chandrasekaran, Somerset, NJ (US); James Garegnani, Somerset, NJ (US)

(73) Assignee: GAVIS PHARMACEUTICALS, Somerset, NJ (US)

(21) Appl. No.: 14/570,901

(22) Filed: Dec. 15, 2014

(51) Int. Cl. A61K 47/38; A61K 9/08; A61K 9/70; A61K 31/4458; A61K 47/32

(52) U.S. Cl. CPC A61K 47/38; A61K 31/4458; A61K 47/32; A61K 9/7007; A61K 9/08

(57) ABSTRACT

An extended release aqueous suspension composition of methylphenidate or salts thereof is provided. The extended release aqueous suspension of methylphenidate or salts has pH of more than 5.0 and exhibits excellent storage stability when tested for impurity and potency of methylphenidate. The suspension also comprises immediate release and sustained release components of methylphenidate or salts thereof. Following administration of a single dose of the extended release aqueous suspension of methylphenidate, a therapeutically effective amount of methylphenidate is reached in less than an hour, provides a release profile of at least 12 hours and has an in vivo release characterized by one single main plasma concentration peak.

### BACKGROUND OF THE INVENTION

(a) Field of the Invention

The present invention is directed to an extended release aqueous suspension composition of methylphenidate or salts thereof. The extended release aqueous suspension of methylphenidate has a pH above 5.0 and exhibits excellent storage stability when tested for impurity and potency. The invention is further directed to the use of said suspension composition for treating a condition susceptible to treatment with methylphenidate. Additionally, the present invention provides a method of manufacture of said suspension composition.

(b) Description of the Related Art

Attention Deficit Disorder (ADD), a commonly diagnosed nervous system illness in children, is generally treated with methylphenidate hydrochloride (available commercially as, e.g., Ritalin™). Symptoms of ADD include distractibility and impulsivity. A related disorder, termed Attention Deficit Hyperactivity Disorder (ADHD), is further characterized by symptoms of hyperactivity, and is also treated with methylphenidate hydrochloride. Methylphenidate has also been used to treat cognitive decline in patients with Acquired Immunodeficiency Syndrome (AIDS) or AIDS related conditions. See, e.g., Brown, G., Anti. J. Psych. Med. 25(1): 21-37 (1995); Holmes et al., J: Clin. Psychiatry 50: 5-8 (1989).

Methylphenidate is used in the form of its hydrochloride (HCl) salt in several commercial products, including, e.g., Ritalin™, Daytrana™, and Metadate™. Methylphenidate HCl It is a racemic mixture of d,1-threo-methyl α-phenyl-2-piperidineacetate hydrochloride and has the empirical formula C₁₄H₁₉NO₂HCl.

The shortcomings of immediate release methylphenidate preparations, including lack of adequate therapy during the day and necessity of administering frequent or multiple dosage forms, particularly in children, has been addressed by various extended or sustained release dosage forms including tablets, capsules and suspensions.

In the prior art, many approaches have been disclosed for making effective extended release dosage forms of methylphenidate.

For example, sustained release formulations of methylphenidate have been developed that provide for slow release of the drug over the course of the day. A further approach resides in the provision of pulsed-release dosage forms, which mimic the effect of prior art medicaments administered at two or more time points during a day. The wash out period provided by the fall off of the plasma concentration of the active ingredient between peaks has been thought to be a contributing factor in reducing or preventing patient tolerance to various types of drugs. Therefrom, it was concluded that some of the therapeutic and pharmacological effects intrinsic in a pulsatile system may be lost or diminished as a result of the constant or nearly constant plasma levels achieved by, e.g., a zero-order release drug delivery systems.

The modified release compositions or formulations which substantially mimics the release of frequent IR dosage regimes, while reducing the need for frequent dosing, thus was said to be more desirable.

Solid dose extended release methylphenidate products are commercially available. These products include, e.g., Concerta™, Ritalin™ LA, and Metadate™.

WO 98/14168 teaches a dosage form providing release of methylphenidate in a sustained and constantly ascending rate. The dosage form disclosed comprises a plurality of beads comprising a hydrogel matrix with increasing amounts of the active ingredient therein, coated with varying amounts of a release rate controlling material. However, WO 98/14168 does not teach achieving a rapid and high main peak plasma concentration, which is desirable in the treatment of, e.g. ADHD.

WO 97/03672 discloses that methylphenidate exhibits a therapeutic effect when administered in the form of a racemic mixture or in the form of a single isomer (such as the RR d-threo enantiomer).

U.S. Pat. No. 5,837,284 discloses a methylphenidate dosage form having immediate release and delayed release particles. The delayed release is provided by the use of ammonio methacrylate pH independent polymers combined with certain fillers and the dosage form is in the form of powder.

WO 2014028610 discloses a methylphenidate extended release chewable tablet containing two immediate release providing components and a barrier coated sustained release providing component.

U.S. Pat. No. 6,228,398 discloses a multiparticulate modified release composition of methylphenidate containing modified release coating or matrix. The composition delivers active ingredient in pulsatile manner.

U.S. Pat. No. 6,344,215 discloses a modified release composition of methylphenidate containing a mixture of IR and ER beads with coating layers of methylphenidate or rate controlling polymers.

U.S. Pat. No. 8,580,301 discloses an enteric coated methylphenidate composition that exhibits one single plasma concentration peak after administration.

Since methylphenidate based medications are predominantly prescribed for children, including children as young as 3 years old where they have difficulty in swallowing the solid dosage forms, a long acting long-acting liquid methylphenidate product which can be conveniently delivered in an oral, titratable formulation was desirable.

Qullivant XR™ is the commercially available oral suspension product of methylphenidate hydrochloride. U.S. Pat. Nos. 8,287,903; 8,465,765; 8,563,033 and 8,778,390 disclose oral methylphenidate extended release powder and aqueous suspension products. The patents further disclose that the suspension formulation of methylphenidate remains stable only when the pH of the suspension is maintained within certain range, particularly below 5.0. According to these patents, when the pH of the suspension products exceeds 5.0, there will be loss of potency and stability of the product.

There remains a need for methylphenidate aqueous suspension formulations having a higher pH and which can remain stable throughout the storage period. Such formulation would advantageously provide freedom of employing a wide range of excipients which are alkaline in nature and contribute in an increase in the formulation pH.

### SUMMARY OF THE INVENTION

The present invention provides methylphenidate extended release aqueous suspensions having a pH that more than 5.0, such as a pH of about 5.1, 5.2, 5.3, 5.4, 5.5 or higher. The suspension further may comprise immediate release and sustained release components of methylphenidate in order to facilitate the desired rapid onset of action and longer therapeutic effect. In an embodiment, the extended release aqueous suspension is prepared by mixing a composition of methylphenidate in the form of a powder comprising immediate release and sustained release components of methylphenidate which then is mixed with water to form an orally administrable extended release aqueous suspension. Also provided is the orally administrable methylphenidate extended release aqueous suspension which is stable at room temperature. Methods of treating patients in need thereof with these methylphenidate extended release suspensions are further provided by the invention.

In one aspect, the invention provides an extended release aqueous suspension of methylphenidate or salts thereof wherein the pH of the suspension is more than 5.0. In an embodiment, the pH of the suspension is about 5.3 or more. In another embodiment, the suspension comprises at least 50% by weight water based on the total weight of the liquid component of the suspension. In a further embodiment, the suspension contains at least about 80% water by weight based on the total weight of the suspension.

In another aspect, the invention provides an extended release aqueous suspension of methylphenidate, preferably in the form of a powder blend, comprising (i) an immediate release methylphenidate component, (ii) a sustained release methylphenidate component, and (iii) an optional water soluble buffering agent. Upon being prepared as an orally administrable aqueous extended release suspension (e.g., reconstituted) formulation, the pH of the suspension is more than 5.0, such as a pH of about 5.1, 5.2, 5.3, 5.4, 5.5 or higher.

In an embodiment, the immediate release and sustained release components in the suspension comprise a methylphenidate-ion exchange resin complex matrix. In another embodiment, the immediate release methylphenidate component is an uncoated methylphenidate ion exchange resin complex matrix, optionally in combination with a matrix forming polymer.

In another embodiment, the sustained release methylphenidate component comprises a methylphenidate ion exchange resin complex matrix along with one or more matrix forming polymers. In an embodiment, the matrix forming polymers are selected from pH-dependent polymer(s), pH-independent polymer(s), or mixtures thereof. In a preferred embodiment, the sustained release methylphenidate component comprises a methylphenidate ion exchange resin complex in a matrix of pH-dependent polymer and devoid of any pH independent polymers. In another preferred embodiment, the sustained release methylphenidate component is devoid of any coating layer.

In another aspect, the extended release aqueous suspension of methylphenidate comprises (i) an immediate release component comprising methylphenidate or salts thereof, (ii) a sustained release component comprising methylphenidate or salts thereof optionally coated with a matrix forming polymer, and (iii) an optional water soluble buffering agent, wherein the pH of the suspension is more than 5.0, such as a pH of about 5.1, 5.2, 5.3, 5.4, 5.5 or higher.

In an embodiment, the matrix forming polymer in the coating comprises pH-dependent polymers, pH independent polymers or mixtures thereof. In another embodiment, the pH-dependent polymers in the sustained release methylphenidate component are selected from acrylic acid polymers, copolymers, phthalates or mixtures thereof.

In another aspect, the extended release aqueous suspension of methylphenidate comprises (i) an immediate release component comprising methylphenidate or salts thereof, (ii) a sustained release component comprising methylphenidate or salts thereof, wherein the sustained release components are coated with separate layers of pH independent polymer(s) and pH dependent polymer(s). In an embodiment, the layers of pH independent polymer(s) and pH dependent polymer(s) are sequentially coated over the methylphenidate-ion exchange resin complex matrix, i.e., applying the pH independent layer first followed by layer of the pH dependent polymer.

In another aspect, the extended release aqueous suspension of methylphenidate comprises (i) an immediate release component comprising methylphenidate or salts thereof, (ii) a sustained release component comprising methylphenidate or salts thereof, wherein the components (i) and (ii) are in the form of particles or granules having an average size range of about 100 microns to about 250 microns, characterized in that pH of the suspension is more than 5.0, such as a pH of about 5.1, 5.2, 5.3, 5.4, 5.5 or higher. Optionally, the extended release aqueous suspension further comprises an optional diluent granule comprising a buffering agent such that upon being formed into an aqueous liquid suspension, the suspension has a pH more than 5.0, preferably about 5.3 or more.

In another aspect, the invention provides an extended release aqueous suspension of methylphenidate or salts thereof wherein the pH of the suspension is more than 5.0 and in vivo release of the composition is characterized by one single main plasma concentration peak.

In another aspect, the invention provides a process for the manufacture of an extended release aqueous suspension of methylphenidate or salts thereof comprising immediate release components and sustained release components of methylphenidate or salts thereof, which process comprises the steps of:
(a) manufacturing the immediate release component by: preparing a methylphenidate-ion exchange resin complex, and optionally granulating the complexes with one or more matrix forming polymers to form granules;
(b) manufacturing the sustained release component by: providing granules manufactured in accordance with step (a), optionally granulating the complexes with one or more matrix forming polymers to form granules, coating the granules with a layer of a pH independent polymer followed by a layer of a pH dependent polymer.

In an embodiment, the process of steps (a) and (b) further comprises mixing the granules with a mixture of one or more excipients including water soluble buffering agents, preservatives, sweeteners, flavours, glidants, etc. to form a powder blend. In an embodiment, separate granules of the excipients are prepared and mixed with granules prepared in steps (a) and (b).

In a further embodiment, the invention provides a method of treating patients with a disorder for which methylphenidate is regulatory approved by administering an oral aqueous methylphenidate extended release suspension formulation as described herein.

In another embodiment, the formulation is an extended release aqueous suspension of methylphenidate that consists essentially of (i) an immediate release component comprising methylphenidate or salts thereof, (ii) a sustained release component comprising methylphenidate or salts thereof optionally coated with a matrix forming polymer, and (iii) a water soluble buffering agent, wherein the pH of the suspension is greater than 5.0, such as a pH of 5.1, 5.2, 5.3, 5.4, 5.5 or higher. The buffering agent may be anhydrous sodium citrate.

In another embodiment, the formulation is an extended release aqueous suspension of methylphenidate that consists essentially of (i) an immediate release component comprising methylphenidate or salts thereof, (ii) a sustained release component comprising methylphenidate or salts thereof optionally coated with a matrix forming polymer, (iii) a water soluble buffering agent, and (iv) an acidic agent to adjust the pH of the suspension to be greater than 5.0, such as a pH of 5.1, 5.2, 5.3, 5.4, 5.5 or higher. The buffering agent may be anhydrous sodium citrate and the acid agent may be anhydrous citric acid in amounts selected to give the desired pH.

In another embodiment, the formulation is an extended release aqueous suspension of methylphenidate that consists of (i) an immediate release component comprising methylphenidate or salts thereof, (ii) a sustained release component comprising methylphenidate or salts thereof optionally coated with a matrix forming polymer, and (iii) a water soluble buffering agent, wherein the pH of the suspension is greater than 5.0, such as a pH of 5.1, 5.2, 5.3, 5.4, 5.5 or greater. The buffering agent may be anhydrous sodium citrate.

In another embodiment, the formulation is an extended release aqueous suspension of methylphenidate that consists of (i) an immediate release component comprising methylphenidate or salts thereof, (ii) a sustained release component comprising methylphenidate or salts thereof optionally coated with a matrix forming polymer, (iii) a water soluble buffering agent, and (iv) an acidic agent to adjust the pH of the suspension to be greater than 5.0, such as a pH of 5.1, 5.2, 5.3, 5.4, 5.5 or greater. The buffering agent may be anhydrous sodium citrate and the acid agent may be anhydrous citric acid in amounts selected to give the desired pH.

Still other aspects and advantages of the invention will be apparent from the following detailed description of the invention.

### DETAILED DESCRIPTION OF THE INVENTION

The invention provides for an extended release aqueous suspension composition of methylphenidate or salts thereof having pH more than 5.0, such as a pH of about 5.1, 5.2, 5.3, 5.4, 5.5 or higher. Preferably, the composition contains, at least, a combination of an immediate release methylphenidate component and a sustained release methylphenidate component.

The invention minimizes stability problems attributed to prior art methylphenidate aqueous suspension formulations having a pH more than 5.0. The immediate and sustained release methylphenidate components in the methylphenidate aqueous suspension of the invention comprises matrix forming polymers admixed with methylphenidate-ion exchange resin complex to form a homogeneous matrix or in the form of a coating over the methylphenidate-ion exchange resin complex matrix. Preferably, the sustained release methylphenidate components are devoid of any coating.

The aqueous suspension of the invention is one in which water is greater than 50% by weight of the liquids in the suspension. In one embodiment, water is greater than about 60%, greater than about 70%, greater than about 80%, greater than about 90%, or up to 100% by weight of the liquid component of the suspension formulation.

The invention, for example, provides for extended release aqueous suspension of methylphenidate having a pH of more than 5.0, such as a pH of about 5.1, 5.2, 5.3, 5.4, 5.5 or higher. In another embodiment, the pH of the suspension is about 5.3 or more. The inventors have observed, in contrast to the prior art teaching, that a peculiar extended release aqueous suspension of methylphenidate if formulated by maintaining the pH of the composition to be more than 5.0, exhibits excellent storage stability when stored at 40° C./75% RH (relative humidity) for at least one month.

The inventors have further observed that a methylphenidate aqueous suspension in accordance with the invention maintains about 98% of its initial potency with the amount of the methylphenidate primary impurity (theo-α-phenyl-1-piperidineacetic acid hydrochloride) limited to not more than 1.0% by weight of the methylphenidate or salt thereof after 4 months of storage of the aqueous suspension at room temperature.

Suitably, following administration of a single dose of the methylphenidate extended release aqueous suspension, in some embodiments, a therapeutically effective amount of methylphenidate is reached as soon as about forty-five minutes and the formulation provides an extended release profile to at least about 12 hours.

As used herein, the term “extended release” refers to compositions which are characterized by having methylphenidate release over a period of at least about 12 hours. As with formulations described herein, “extended release” may be achieved by a single formulation containing both an “immediate release” component (release in less than one hour, e.g., as soon as about 45 minutes or as soon as about 30 minutes after administration) and a “sustained release” component (i.e., release for about 12 hours after administration). The release profile may be assessed via in vitro dissolution using techniques known to those of skill in the art [e.g., USP basket method, Paddle Method, channel flow method, or other methods known in the literature]. The release profile can be assessed in vivo (e.g., for bioavailability determinations), using plasma concentrations to assess maximum plasma concentration (Cₘₐₓ) and area under the curve (AUC). Such assays are well known to those of skill in the art. [see, e.g., W. Wargin, et al., Pharmacokinetics of methylphenidate in man, rat and monkey. J Pharmacol Exp Ther August 1983 226:382-386]. Bioequivalence is established by comparing pharmacokinetic parameters, for example AUC and Cₘₐₓ, of the present invention with the Quillivant® XR suspension in healthy human subjects. The term “AUC” refers to the area under the time/plasma concentration curve after the administration of the methylphenidate extended-release suspension dosage form to healthy human subjects. The term “Cₘₐₓ” refers to the maximum concentration of methylphenidate in the blood following the administration of the methylphenidate extended-release suspension dosage form to healthy human subjects. To show bioequivalence, the 90% confidence interval of the AUC and Cₘₐₓ values of the test product should be within a range of 80% to 125% of the reference product.

As used herein, a “methylphenidate-ion exchange resin complex or particle” is a methylphenidate containing ion-exchange resin particle in which there is an ionic bond between methylphenidate and the ion-exchange resin particle.

The term “immediate release” is the release of methylphenidate from a formulation where the rate of release of methylphenidate from the formulation is not retarded by means of a controlled release matrix or other such means and where the components of the formulation are designed such that, upon ingestion, maximum exposure of said drug to body tissues occurs in the minimum period of time. As described herein, an “immediate release” methylphenidate component preferably releases in less than 1 hour, e.g., as soon as about 45 minutes or as soon as about 30 minutes after administration. Further, in one embodiment, the methylphenidate immediate release component releases at least about 50% of the methylphenidate within about the first hour following administration, and at least about 80% of the methylphenidate within about 90 minutes following administration. As described further, a methylphenidate ion exchange resin complex, optionally in a matrix, and may provide the immediate release component.

As used herein “methylphenidate” includes the active ingredient which is either (i) a racemic mixture of two optical isomers, d-threo-methylphenidate and 1-threo-methylphenidate, or (ii) the active isomer d-threo-methylphenidate (also known as dexmethylphenidate). Where only the racemate or dexmethylphenidate is desired, reference will be specifically made to one or the other. Thus, for the formulations described herein, the methylphenidate may be independently selected from racemic methylphenidate (e.g., a 50/50 mixture of D-methylphenidate and L-methylphenidate), and dexmethylphenidate.

The therapeutically effective amount of methylphenidate is at least the minimum amount of methylphenidate which reduces or eliminates the symptoms associated with a condition for which methylphenidate has been approved for use.

In one embodiment, the extended release aqueous suspension comprises at least 50% by weight water based on the total weight of the liquid component of the suspension, wherein the pH of the suspension is more than 5.0. In another embodiment, the pH of the suspension is about 5.3 or more. In another embodiment, the suspension contains at least about 80% water by weight based on the total weight of the suspension.

The extended release aqueous suspension of methylphenidate is preferably in the form of a powder blend that includes (i) an immediate release methylphenidate component, (ii) a sustained release methylphenidate component, and (iii) an optional water soluble buffering agent. Upon being prepared as an orally administrable aqueous extended release suspension (e.g., reconstituted) formulation, the pH of the suspension is more than 5.0, such as a pH of about 5.1, 5.2, 5.3, 5.4, 5.5 or higher.

In an embodiment, the immediate release and sustained release components in the suspension comprise a methylphenidate-ion exchange resin complex matrix. In another embodiment, the immediate release methylphenidate component is an uncoated methylphenidate ion exchange resin complex matrix, optionally in combination with a matrix forming polymer.

In an embodiment, the sustained release methylphenidate component comprises a methylphenidate ion exchange resin complex matrix along with one or more matrix forming polymers. In an embodiment, the matrix forming polymers are selected from pH-dependent polymer(s), pH-independent polymer(s), or mixtures thereof. In a preferred embodiment, the sustained release methylphenidate component comprises a methylphenidate ion exchange resin complex in a matrix of pH-dependent polymers and devoid of any pH independent polymers. In another preferred embodiment, the sustained release methylphenidate component is devoid of any coating layer.

In another aspect, the extended release aqueous suspension of methylphenidate comprises (i) an immediate release component comprising methylphenidate or salts thereof, (ii) a sustained release component comprising methylphenidate or salts thereof optionally coated with a matrix forming polymer, and (iii) an optional water soluble buffering agent, wherein the pH of the suspension is more than 5.0, such as a pH of about 5.1, 5.2, 5.3, 5.4, 5.5 or higher.

In an embodiment, the matrix forming polymer(s) in the coating comprises pH-dependent polymer(s), pH independent polymer(s) or mixtures thereof. In another embodiment, the pH-dependent polymer(s) in the sustained release methylphenidate component are selected from acrylic acid polymers, copolymers, phthalates or mixtures thereof.

In another embodiment, the extended release aqueous suspension of methylphenidate comprises (i) an immediate release component comprising methylphenidate or salts thereof, and (ii) a sustained release component comprising methylphenidate or salts thereof, wherein the sustained release components are coated with separate layers of pH independent polymer(s) and pH dependent polymer(s). Preferably, the layers of the pH independent polymer(s) and the pH dependent polymer(s) are sequentially coated over the methylphenidate-ion exchange resin complex matrix, i.e., applying a pH independent layer first and followed by layer of pH dependent polymer(s).

In another embodiment, the extended release aqueous suspension of methylphenidate comprises (i) an immediate release component comprising methylphenidate or salts thereof, (ii) a sustained release component comprising methylphenidate or salts thereof, wherein the components (i) and (ii) are in the form of particles or granules having an average size range of about 100 microns to about 250 microns, characterized in that the suspension has pH more than 5.0, such as a pH of about 5.1, 5.2, 5.3, 5.4, 5.5 or higher. Optionally, the extended release aqueous suspension further comprises an optional diluent granule comprising a buffering agent such that upon being formed into an aqueous liquid suspension, the suspension has pH more than 5.0, preferably about 5.3 or more.

In another embodiment, the extended release aqueous suspension of methylphenidate or salts thereof wherein the pH of the suspension is more than 5.0 and in vivo release of the composition is characterized by one single main plasma concentration peak.

The term “component” refers to particles, granules, pellets, tablets or mini-tablets comprising methylphenidate-ion exchange resin complex optionally in a matrix of one or more pharmaceutically acceptable excipients.

The term “methylphenidate-ion exchange resin complex” refers to the product resulting from loading a methylphenidate salt onto a cation exchange resin. Methods for preparing such complexes have been described in various references. For example, WO 2007/109104, incorporated herein by reference, describes a method of forming a drug-ion exchange resin complex by mixing the drug and the ion exchange resin together in an aqueous medium to facilitate the “exchange” between the salt of the methylphenidate and the “cation” of the ion exchange resin and the formation of the complex.

A “methylphenidate-ion exchange resin complex matrix” also refers to a methylphenidate-ion exchange resin complex which may be further combined, e.g., prior to or during granulation, with a polymeric material which forms a matrix with the methylphenidate-ion exchange resin complex. In one embodiment, a “methylphenidate polistirex” refers to the complex (salt) formed by loading methylphenidate onto an ion exchange resin.

Resins suitable for complexation with methylphenidate or salts thereof are cationic exchange resins. Examples of cation exchange resins include, but are not limited to, a sulfonated copolymer of styrene and divinyl benzene (available commercially under the brand Amberlite® IRP69), cross-linked polymer of methacrylic acid and divinyl benzene (available commercially under brand Amberlite® IRP88), methacrylic acid and divinyl benzene polymer (hydrogen ion) polyacrilex resin (available commercially under brand Amberlite® 64), a weakly acidic (potassium ion) cation exchange resin with 4% cross-linked methacrylate and Dowex® 50WX8.

The amount of methylphenidate that can be complexed with a resin typically ranges from about 5% to about 50% by weight of the methylphenidate-ion exchange resin complex particles. In one embodiment, loading of about 10% to about 40% by weight, more desirably, about 15% to about 30% by weight, or about 25% of the methylphenidate-ion exchange resin complex particles can be employed.

After complexation, the methylphenidate-ion exchange resin complex particles may be milled to achieve a desired size range, dried, and then stored for future use. In one embodiment, the complex is milled or passed through a sieve to provide a particle size ranging from about 40 microns to about 410 microns to enhance mouth feel. These particles may be either regularly or irregularly shaped. In some embodiments, the average particle size of the methylphenidate-ion exchange resin complex is milled to a size of about 100 to about 200 microns.

The methylphenidate-ion exchange resin complex particles or methylphenidate-ion exchange resin complex matrix, which may be prepared by any suitable method known in the art, are used as the immediate release component. In an embodiment, the immediate release component further may contain one or more matrix forming polymers to facilitate granulation, the amount of which however is maintained to the extent that immediate release from such component is not affected. The matrix forming polymers in such case may be present either in the matrix, in the form of a coating or a combination of both.

The immediate release component of methylphenidate-ion exchange resin or methylphenidate-ion exchange resin complex matrix may be used for preparing sustained release components. Suitable matrix forming polymers are used in the sustained release components in order to modify release of the drug from such component. In an embodiment, the matrix forming polymers are present in the matrix along with the methylphenidate-ion exchange resin complex matrix. In a further embodiment, the rate controlling polymers are present in the form of coating over the methylphenidate-ion exchange resin complex matrix. In a further embodiment, the matrix forming polymers in the methylphenidate-ion exchange resin complex matrix are present in the form of a coating as well as a matrix.

The coating layer of matrix forming polymers may be applied as an aqueous suspension over the immediate release methylphenidate component and form a separate layer thereon. Preferably, the coating layer is applied directly over the immediate release methylphenidate component, i.e., there are no intervening layers between the coat and immediate release methylphenidate component. Depending upon the polymeric material selected, the coat layer may be cured.

Matrix forming polymers which are used to prepare immediate release and/or sustained release components include pH dependent polymers, pH independent polymers or mixtures thereof. In an embodiment, the matrix of the methylphenidate-ion exchange resin complex and the pH-dependent polymer(s) may be used to prepare an extended release aqueous suspension of the present invention, particularly to form sustained release methylphenidate components.

Suitable matrix forming polymers include either pH dependent polymers/co-polymers, pH independent polymers/co-polymers or mixtures thereof which form a matrix with the methylphenidate-ion exchange resin complex upon being admixed or granulated therewith.

Examples of matrix forming polymers include, but are not limited to polyvinyl acetate polymers or a mixture of polymers containing same (e.g., KOLLICOAT™ SR 30D), cellulose acetates, ethylcellulose polymers (e.g., AQUACOAT™ ECD-30 or SURELEASE™), acrylic based polymers or copolymers (e.g., represented by the EUDRAGIT family of acrylic resins), cellulose phthalate, or any combination of such water-insoluble polymers or polymer systems. The preferred matrix forming polymers include, but are not limited to polymers or copolymers of acrylic acid, including (co)polymers of (meth)acrylic acid and/or (meth)acrylate which are available commercially under the brand name EUDRAGIT; and phthalates include cellulose acetate phthalate, hydroxypropyl methylcellulose phthalate, hydroxypropyl methylcellulose acetate succinate) and polyvinyl acetate phthalate. Examples of suitable acrylic polymers from the EUDRAGIT family may include, e.g., a copolymer comprising ethyl acrylate and methyl methacrylate (e.g., EUDRAGIT NE-30D), or EUDRAGIT RS, RL30D, RL100, or NE, which are largely pH-independent polymers; although less desirable, certain pH-dependent members polymers including, e.g., members of the EUDRAGIT polymer family, e.g., the L, S, and E, polymers may be selected.

The term “(co)polymers of (meth)acrylic acid and/or (meth)acrylate” comprises all polymers and copolymers based on methacrylic acid and acrylic acid as well as their derivatives. For example, by “(meth)acrylate” monomers is meant derived from methacrylic acid and/or acrylic acid (i.e., methacrylic esters and acrylic esters, methacrylic hydroxyalkyl esters and acrylic hydroxyalkyl esters, etc.). Furthermore, the term also encompasses polyacrylate polymers.

The quantity of matrix forming polymer that is added to the immediate release methylphenidate component as a matrix or coated as layer typically ranges from about 1% to about 30%, or about 3% to about 20%, or about 3% to about 10% or more by weight of the methylphenidate-ion exchange resin complex matrix particulates. Following admixing the immediate release component particles with the matrix forming polymer, the resulting mixture is dried and the immediate release component granules then are milled appropriately to the desired particulate size.

The sustained release component of a methylphenidate extended release powder blend of one aspect of the invention contains a methylphenidate-ion exchange resin complex matrix or immediate release methylphenidate component with a coating which modifies the release profile of the immediate release component such that the methylphenidate has about a twelve hour sustained release profile. In one embodiment, the coating layer is about 10% to about 70% by weight, or about 15% to about 65% by weight, of the methylphenidate ion exchange resin complex matrix in order to provide the sustained release component.

In one embodiment, the coating is applied as an aqueous dispersion which is dried and cured in order to provide the desired sustained release profile (e.g., polyvinylacetate or ethylcellulose-based coatings). Such a cured coating layer may be in the range of about 15% by weight to about 70% by weight, or about 20% by weight to about 60% by weight, or about 30% by weight to about 45% by weight, based on the total weight of the methylphenidate-ion exchange resin complex matrix.

Generally, a plasticizer is used in the percent range, or a mixture of plasticizers combine to total about 2% to about 50% by weight of the coating layer, more preferably about 2.5% to about 20% by weight of the coating layer on the coated methylphenidate-ion exchange resin complex. Preferably a plasticizer is in the range of about 2.5% to about 15% by weight of the coating layer based on the coated complex providing the most desirable properties. Suitable plasticizers may be water soluble and water insoluble. Examples of suitable plasticizers include dibutyl sebacate, propylene glycol, polyethylene glycol, polyvinyl alcohol, triethyl citrate, acetyl triethyl citrate, acetyl tributyl citrate, tributyl citrate, triacetin, and Soluphor™ P (2-pyrrolidone), and mixtures thereof.

In one embodiment, the coating may be a EUDRAGIT™ brand acrylate based coating material [including, e.g., a poly (ethyl acrylate-co-methyl methacrylate-co-trimethylammonioethyl methacrylate chloride) polymer system]. For example, Eudragit™ RS 30D or Eudragit™ RE 30D may be selected for coating. In one embodiment, a blend of Eudragit™ RS 30D and Eudragit™ RE 30D may be prepared to optimize the hydrophilicity/hydrophobility of the film.

In order to achieve the desired suspension composition, the immediate release and sustained release methylphenidate components are prepared in the form of a powder blend. In one embodiment, the blend contains about 5% to about 30%, or about 10% to about 25%, or about 20% by weight of the immediate release methylphenidate component to about 70% to about 95%, about 75% to about 90%, by weight, or about 80% by weight of the sustained release methylphenidate component, based on the total weight of the methylphenidate.

In one embodiment, the powder blend also contains a diluent granule, which facilitates reconstitution of immediate release components, sustained release methylphenidate components and optionally also provides agents for improving the flow of the powder (e.g., glidants), sweeteners or other flavourings, or suspending agents.

In one embodiment, a diluent granule used in the invention contains a buffering species used to control pH in the liquid suspension formulation. Optionally, the diluent granule may contain one or more other excipients including, e.g., a glidant, a flavoring agent, a preservative, a suspending agent, or mixtures of such excipients.

Suitably, the buffering species are selected so that upon being combined with water and any other components of a placebo suspension base, the final oral aqueous liquid suspension formulation has a pH of more than 5.0, or about 5.3 or more. Examples of buffering agents include, but are not limited to acid selected from citric acid, ascorbic acid, acetic acid, tartaric acid, phosphoric acid or salts thereof.

Suitable sweeteners which may be used to prepare the suspension composition of the present invention may be chosen from water-soluble sweetening agents such as monosaccharides, disaccharides and polysaccharides such as xylose, ribose, glucose, mannose, galactose, fructose, high fructose corn syrup, dextrose, sucrose, sugar, maltose, partially hydrolyzed starch, and corn syrup solids and sugar alcohols such as sorbitol, xylitol, mannitol and mixtures thereof.

Suitable flavorings include both natural and artificial flavors, and mints such as peppermint, menthol, artificial vanilla, cinnamon, various fruit flavors, both individual and mixed, essential oils (i.e., thymol, eucalyptol, menthol and methyl salicylate) and the like are contemplated.

Useful preservatives include, but are not limited to, sodium benzoate, benzoic acid, potassium sorbate, salts of edetate (also known as salts of ethylenediaminetetraacetic acid, or EDTA, such as disodium EDTA), parabens (e.g., methyl, ethyl, propyl or butyl-hydroxybenzoates, etc.), sorbic acid, and chelating agents.

Optionally, these diluent granules as described herein may form part of the methylphenidate extended release powder blend formulation. When present, the diluent granules may be in an amount of about 1% to about 90% by weight, or about 10% to about 85% by weight, or about 50% to about 75% by weight of the total methylphenidate extended release powder blend.

The ratio of immediate release methylphenidate component to sustained release methylphenidate component may be adjusted as desired by one of skill in the formulation art. In one embodiment, the powder blend contains about 5 to about 20 parts, about 95 to about 80 parts, about 10 to about 30 parts, or about 90 to about 70 parts by weight of immediate and sustained release methylphenidate components.

In one embodiment, the invention provides a methylphenidate aqueous extended release oral suspension comprising at least 50% by weight water based on the total weight of the liquid component of the suspension, wherein extended release is as defined herein (e.g., provides a therapeutically effective plasma profile for about 12 hours). In one embodiment, the suspension contains at least about 80% by weight water based on the total weight of the suspension. In one embodiment, the pH of the suspension is more than 5.0. In another embodiment, pH of the suspension is about 5.3 or more.

The invention further provides a method for the manufacture of an extended release aqueous suspension of methylphenidate or salts thereof. The method comprises the following steps:
(a) manufacturing the immediate release component by: preparing a methylphenidate-ion exchange resin complex, and optionally granulating the complexes with one or more matrix forming polymers to form granules.
(b) manufacturing the sustained release component by: providing granules manufactured in accordance with step (a), optionally granulating the complexes with one or more matrix forming polymers to form granules, coating the granules with a layer of pH independent polymer followed by a layer of pH dependent polymer.

In an embodiment, the process of steps (a) and (b) further comprises mixing the granules with a mixture of one or more excipients including water soluble buffering agents, preservatives, sweeteners, flavours, glidants, etc. to form a powder blend. In an embodiment, separate granules of the excipients are prepared and mixed with granules prepared in steps (a) and (b). The powder blend is then mixed with purified water in a predetermined amount to prepare a suspension suitable for oral administration.

The aqueous methylphenidate extended release suspension of the invention may be orally administered to a patient having a disorder treatable by methylphenidate. These include disorders for which regulatory approval has been obtained. For example, methylphenidate is currently approved for treatment of ADHD, postural orthostatic tachycardia syndrome, narcolepsy, behavioral disorders, depression and psychiatric disorders.

### Example 1

### Methylphenidate-Ion Exchange Resin Particles

TABLE 1

| Sr. | | Quantity |
|---|---|---|
| No. | Ingredient | (gm) |
| 1 | Methylphenidate HCl | 500 |
| 2 | Sodium polystyrene sulfonate (Amberlite IRP 69) | 1500 |
| 3 | Purified Water | 4700 |

| | |
|---|---|
| Total | 6700 |

Procedure:

Methylphenidate HCl was dissolved in purified water under stirring to form a clear solution. Sodium polystyrene sulfonate (resin) was added to the methylphenidate HCl solution and stirred for 4 hours. The dispersion was then filtered or centrifuged to separate the methylphenidate-ion exchange resin particles. The methylphenidate-resin particles were dried in a tray drier until reaching a moisture content of 3-5% and screened through a Quadro comill fitted with a 40 mesh screen.

### Example 2

### Methylphenidate HCl Immediate Release Components

TABLE 2

| Sr. | | Quantity |
|---|---|---|
| No. | Ingredient | (gm) |
| 1 | Methylphenidate-Ion Exchange Resin particles of | 600 |
| | Example 1 | |
| 2 | Povidone K30 | 480 |
| 3 | Purified Water | 190 |

| | |
|---|---|
| Total | 1270 |

Procedure:

Povidone K30 was dissolved in water under stirring to form a clear solution. The methylphenidate-resin particles of Example 1 were granulated using a rapid mixer granulator with Povidone K30 solution. Wet granules were dried in a tray drier until reaching a moisture content of 3-5% and the dried granules then were screened through a Quadro comill fitted with a 40 mesh screen to form the methylphenidate HCl immediate release components.

### Example 3

### Methylphenidate HCl Sustained Release Components 1

TABLE 3

| Sr. | | Quantity | Quantity |
|---|---|---|---|
| No. | Ingredient | (gm) | (gm) |
| 1 | Methylphenidate IR components | 400 | 400 |
| | as per Example 2 | | |
| 2 | Ethylcellulose 45 cps | 61.7 | — |
| 3 | Ethylcellulose 100 cps | — | 61.7 |
| 4 | Triacetin | 4.1 | 4.1 |
| 5 | Isopropyl Alcohol | 930 | 1421 |
| 6 | Purified Water | 102 | 158 |

| | | |
|---|---|---|
| Total | 1497.8 | 2044.8 |

Procedure:

Isopropyl alcohol and purified water were mixed in a SS container. Ethylcellulose 45 or 100 cps was then added to the solvent mixture and mixed well to form a clear solution. Triacetin was added to the mixture and mixed for 30 minutes. The methylphenidate IR components as per Example 2 were then coated with the dispersion with an exhaust temperature of 30-35° C., a nozzle air pressure of 20-30 psi. The coated granules were then dried in a tray drier at 50° C. for 12 hours and screened through a 40 mesh screen to form the methylphenidate HCl sustained release components.

### Example 4

### Methylphenidate HCl ER Oral Suspension 25 mg/5 ml

TABLE 4

| Sr. | | Quantity | Quantity |
|---|---|---|---|
| No. | Ingredient | (gm) | (gm) |
| 1 | Methylphenidate SR components 2 as per | 124 | 124 |
| | Example 3 | | |
| 2 | Xanthan Gum | 15 | 15 |
| 3 | Talc | 10 | 10 |
| 4 | Silicon Dioxide (Syloid 244 FP) | 10 | 10 |
| 5 | Banana Flavour (Natural & Artificial) | 8 | 8 |
| 6 | Pregelatinised Starch (Starch 1500) | 100 | 100 |
| 7 | Sucrose (Extra fine granulated) | 667.5 | 668.4 |
| 8 | Sodium benzoate | 12 | 12 |
| 9 | Sodium citrate, anhydrous | 41.3 | 38.7 |
| 10 | Anhydrous citric acid | 8.6 | 10.3 |
| 11 | Sucralose | 3.6 | 3.6 |
| 12 | Purified Water | 50 | 50 |

| | | |
|---|---|---|
| Total | 1000 | 1000 |

Procedure:

Ingredients 7 to 11 were mixed and granulated using purified water in a rapid mixer granulator. The wet mass was dried in a tray drier to a moisture content of 2-3% w/w. The dried granules were milled using a Quadro comill fitted with a 40 mesh screen. The milled granules were then mixed with ingredients 1 to 6 in V-blender for 20 minutes. The blended mixture was filled into amber coloured glass or PET bottles. The powder was reconstituted with purified water to produce a palatable suspension. The pH of the suspension measured was 5.3-5.5.

What is claimed is:

1. An extended release aqueous suspension comprising methylphenidate or salts thereof, wherein the pH of the suspension is greater than 5.0.

2. The suspension of claim 1, wherein the pH of the suspension is about 5.3 to about 5.5.

3. The suspension of claim 1, wherein the suspension comprises (i) an immediate release component comprising methylphenidate or salts thereof; (ii) a sustained release component comprising methylphenidate or salts thereof; (iii) an aqueous vehicle; and (iv) an optional water soluble buffering agent.

4. The suspension of claim 3, wherein the immediate release component is in the form of an uncoated methylphenidate ion exchange resin complex, optionally in combination with a matrix forming polymer.

5. The suspension of claim 3, wherein the sustained release component comprises a matrix of a methylphenidate ion exchange resin complex and a matrix forming polymer.

6. The suspension of claim 3, wherein the matrix forming polymers are selected from the group consisting of pH-dependent polymers, pH-independent polymers, and mixtures thereof.

7. The suspension of claim 3, wherein the sustained release component further comprises one or more coating layer(s) of a matrix forming polymer(s).

8. The suspension of claim 7, wherein the coating over the sustained release component comprises a first layer of a pH independent polymer(s) and a second a layer of a pH dependent polymer(s).

9. The suspension of claim 6, wherein the pH-dependent polymers are selected from the group consisting of polymers or copolymers of acrylic acid, cellulose acetate phthalate, hydroxypropyl methylcellulose phthalate, hydroxypropyl methylcellulose acetate succinate), polyvinyl acetate phthalate, and mixtures thereof.

10. The suspension of claim 6, wherein the pH-independent polymers are selected from the group consisting of polyvinyl acetate, cellulose acetates, ethylcellulose and mixtures thereof.

11. The suspension of claim 3, wherein the sustained release component is devoid of a pH independent polymer and/or the immediate release component and the sustained release component are devoid of any coating layer.

12. The suspension of claim 1, wherein the suspension maintains at least about 98% of an initial potency of methylphenidate when stored at 40° C./75% RH for at least one month and maintains the amount of theo-α-phenyl-1-piperidineacetic acid hydrochloride to be not more than 1.0% by weight of methylphenidate or salt thereof after four months of storage of the suspension at room temperature.

13. The suspension of claim 1, wherein the in vivo release of the composition is characterized by a single main plasma concentration peak.

14. The suspension of claim 1, wherein the extended release aqueous suspension of methylphenidate consists essentially of (i) an immediate release component comprising methylphenidate or salts thereof, (ii) a sustained release component comprising methylphenidate or salts thereof optionally coated with a matrix forming polymer, and (iii) a water soluble buffering agent, wherein the pH of the suspension is greater than 5.0.

15. The suspension of claim 1, wherein the extended release aqueous suspension of methylphenidate consists essentially of (i) an immediate release component comprising methylphenidate or salts thereof, (ii) a sustained release component comprising methylphenidate or salts thereof optionally coated with a matrix forming polymer, (iii) a water soluble buffering agent, and (iv) an acidic agent present in an amount to adjust the pH of the suspension to be greater than 5.0.

16. The suspension of claim 1, wherein the extended release aqueous suspension of methylphenidate consists of (i) an immediate release component comprising methylphenidate or salts thereof, (ii) a sustained release component comprising methylphenidate or salts thereof optionally coated with a matrix forming polymer, and (iii) a water soluble buffering agent, wherein the pH of the suspension is greater than 5.0.

17. The suspension of claim 1, wherein the extended release aqueous suspension of methylphenidate consists of (i) an immediate release component comprising methylphenidate or salts thereof, (ii) a sustained release component comprising methylphenidate or salts thereof optionally coated with a matrix forming polymer, (iii) a water soluble buffering agent, and (iv) an acidic agent to present in an amount to adjust the pH of the suspension to be greater than 5.0.

18. A method of manufacturing the suspension of claim 3, wherein the process comprises the steps of:
(a) preparing the immediate release component by: preparing a methylphenidate-ion exchange resin complex, and optionally granulating the complexes with one or more matrix forming polymer to form granules; and
(b) preparing the sustained release component by: providing granules manufactured in accordance with step (a), optionally granulating the complexes with one or more matrix forming polymer to form granules, and coating the granules with a layer of pH independent polymer followed by a layer of pH dependent polymer.

19. The method of claim 18, wherein the process further comprises:
(c) preparing granules of one or more excipients and mixing it with granules prepared in step (a) and (b) to form a powder blend, and
(d) mixing the powder blend in purified water to form a suspension.

20. A method for treating a patient having a condition susceptible to treatment with methylphenidate, the method comprising administering to the patient an extended release aqueous suspension comprising methylphenidate or salts thereof, wherein the pH of the suspension is greater than 5.0.
